(19) United States
(12) Patent Application Publication
(10) Pub. No.: US 2024/0282429 A1
(43) Pub. Date: Aug. 22, 2024

(54) DRINKING AMOUNT MANAGEMENT METHOD, SYSTEM, TERMINAL DEVICE, PROGRAM

(71) Applicant: CureApp, Inc., Tokyo (JP)

(72) Inventor: Ryuhei SO, Tokyo (JP)

(21) Appl. No.: 18/561,155

(22) PCT Filed: Mar. 7, 2022
(86) PCT No.: PCT/JP2022/009816

§ 371 Date: Nov. 15, 2023

(30) Foreign Application Priority Data
May 19, 2021 (JP) 2021-084840

(51) Int. Cl. G16H 20/70; G16H 50/70

(52) U.S. Cl. CPC G16H 20/70; G16H 50/70

(57) ABSTRACT

To provide an improved amount-of-drinking management system and the like. A method performed on a system that includes a user terminal and a management server configured to be connectable from the user terminal, the method including: accepting information about drinking of a user on the user terminal; transmitting the information accepted on the user terminal to the management server; aggregating and/or analyzing information received from the user terminal, on the management server; and feeding back results of the aggregation and/or the analysis to the user terminal, in which the information about the drinking of the user includes at least information about an amount of drinking of the user and context information.

### TECHNICAL FIELD

The present invention relates to a method and the like for managing conditions of diseases and ailments, including diseases highly associated with lifestyle and mental disorders, such as alcohol dependency, that can be treated through behavioral modifications, and more particularly, to a method and the like for managing amounts of drinking, mental condition, and the like of heavy drinkers including alcohol-dependent patients.

### BACKGROUND ART

Conventionally, so-called behavioral modification approaches have been considered to be effective for diseases and ailments, including diseases associated with lifestyle and mental disorders, such as alcohol dependency, that can be treated through behavioral modifications. Specifically, such approaches include an approach whereby the patient, healthcare workers, and people concerned around the patient analyze causes and the like of behavior undesirable for the patient and encourage the patient to change his/her behavior.

A technique has been proposed for managing a person's health or finding physical abnormalities or their signs by paying attention widely to the person's actions, utterances, emotional changes, and the like. For example, a technique for determining a user's state of health based on selection button input information corresponding to an action of the user and multiple emotions of the user has been proposed (Patent Literature 1).

In other words, Patent Literature 1 discloses a determination apparatus comprising: an acquisition unit adapted to acquire selection information that indicates a button selected by a user from among multiple buttons corresponding to a predetermined action of the user and corresponding to respective ones of multiple emotions; and a determination unit adapted to determine a state of health of the user based on the selection information acquired by the acquisition unit.

Another system has been proposed that reliably acquires information about a chief complaint of a subject such as a patient, prevents signs or symptoms of the subject's physical abnormalities from being overlooked, and moreover, provides an ability to find the subject's slight physical abnormalities or their signs (Patent Literature 2).

In other words, Patent Literature 2 discloses a conversation recording system comprising: a voice recording unit adapted to record voice information on conversations carried out among a plurality of speakers including those who are involved in medical service, caregiving, or nursing and subjects who receive medical diagnosis, caregiving, or nursing; a speaker identification unit adapted to identify individual speakers based on analysis of the voice information or on preregistered voice data of the speakers; a voice recognition unit adapted to convert the voice information into text data of the individual speakers by voice recognition; a dialogue sentence creation unit adapted to rearrange the text data of the individual speakers into dialogue form among the plurality of speakers and thereby create sentence data in the dialogue form; and a database adapted to store the sentence data in the dialogue form by associating the sentence data with health management information related to medical service, caregiving, or nursing of each of the subject included in the plurality of speakers such that the sentence data and the health management information are sharable among external accesses.

A technique has also been proposed for accepting input of state-of-health information, which is information about a state of health of a user, extracting state-of-health-related keywords from the accepted state-of-health information, and determining physical condition of the user based on the keywords (Patent Literature 3).

In other words, Patent Literature 3 discloses a health management system that manages health of a user by being equipped with a user terminal, which is a terminal device used by the user placed under health management, and a server device connected to the user terminal and adapted to manage health of the user, the health management system comprising: a state-of-health information input unit adapted to accept input of state-of-health information, which is information about a state of health of the user; a keyword extraction unit adapted to extract state-of-health-related keywords from the state-of-health information accepted by the state-of-health information input unit; a keyword presentation unit adapted to present information containing the keywords extracted by the keyword extraction unit to the user; and a physical condition determination unit adapted to determine physical condition of the user based on the keywords extracted by the keyword extraction unit.

### CITATION LIST

### Patent Literature

Patent Literature 1: Japanese Patent Laid-Open No. 2019-164737
Patent Literature 2: Japanese Patent Laid-Open No. 2018-206055
Patent Literature 3: Japanese Patent Laid-Open No. 2016-224784

### SUMMARY OF INVENTION

### Technical Problem

However, in the techniques disclosed in Patent Literatures 1 to 3, no analysis or determination is conducted by paying attention to relevancy between drinking and health of a user or a patient. Besides, heavy drinking is an undesirable behavior and it is considered to be effective to monitor amounts of drinking in relation to the heavy drinking behavior and analyze resulting information by sharing the information among the patient himself/herself, people concerned with the patient, and healthcare workers. Hitherto, in the field of clinical psychology, cognitive-behavioral therapies based on functional analysis have been tried, but no sufficient proposal has been made yet in terms of a specific system solution to alcohol dependency.

### Solution to Problem

Thus, an amount-of-drinking management method according to an embodiment of the present invention is a method performed on a system that includes a user terminal and a management server configured to be connectable from the user terminal, the method comprising: accepting information about drinking of a user on the user terminal; transmitting the information accepted on the user terminal to the management server; aggregating and/or analyzing information received from the user terminal, on the management server; and feeding back results of the aggregation and/or the analysis to the user terminal, wherein the information about the drinking of the user includes at least information about an amount of drinking of the user and context information.

The context information includes at least one item of information out of: with whom, where, with what feeling, when do you start drinking, and under what circumstances.

### Advantageous Effects of Invention

The amount-of-drinking management method and the like according to an embodiment of the present invention achieve the effect of being able to more appropriately provide feedback about reduction of drinking, improvement of physical condition, and furthermore, promotion of health to the user.

### BRIEF DESCRIPTION OF DRAWINGS

FIG. 1 is an explanatory diagram illustrating an exemplary overall configuration of an amount-of-drinking management system according to an embodiment of the present invention.

FIG. 2 is an explanatory diagram illustrating a functional block configuration of a management server in the amount-of-drinking management system according to an embodiment of the present invention.

FIG. 3 is an explanatory diagram illustrating an exemplary external appearance configuration of an information processing apparatus (user terminal, doctor's terminal, or the like) in the amount-of-drinking management system according to an embodiment of the present invention.

FIG. 4 is an explanatory diagram illustrating a functional block configuration of hardware making up the information processing apparatus according to an embodiment of the present invention.

FIG. 5 is an explanatory diagram illustrating a flow of processing operations in the amount-of-drinking management system according to an embodiment of the present invention.

FIG. 6 is an explanatory diagram illustrating a flow of processing operations in the amount-of-drinking management system according to an embodiment of the present invention.

FIG. 7 is a flowchart illustrating a processing operation flow in the amount-of-drinking management system according to an embodiment of the present invention.

FIG. 8 is a flowchart illustrating a processing operation flow in the amount-of-drinking management system according to another embodiment of the present invention.

FIG. 9 is a flowchart illustrating a processing operation flow in the amount-of-drinking management system according to an embodiment of the present invention.

FIG. 10 is an explanatory diagram illustrating a display example of the information processing apparatus in the amount-of-drinking management system according to an embodiment of the present invention.

FIG. 11 is an explanatory diagram illustrating a display example of the information processing apparatus in the amount-of-drinking management system according to an embodiment of the present invention.

FIG. 12 is an explanatory diagram illustrating a display example of the information processing apparatus in the amount-of-drinking management system according to an embodiment of the present invention.

FIG. 13 is an explanatory diagram illustrating a display example of the information processing apparatus in the amount-of-drinking management system according to an embodiment of the present invention.

### DESCRIPTION OF EMBODIMENT

An amount-of-drinking management system and the like according to an embodiment of the present invention will be described in detail below with reference to the accompanying drawings.

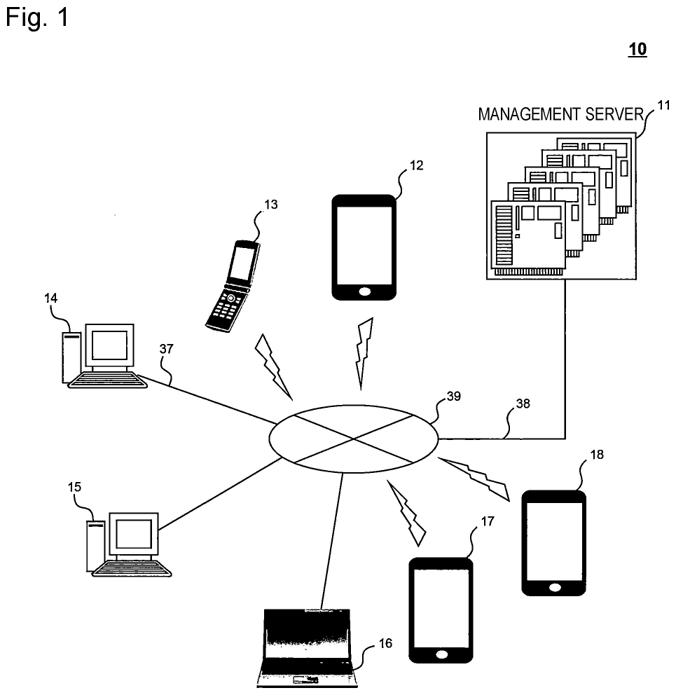

An exemplary overall configuration of the amount-of-drinking management system according to an embodiment of the present invention is shown in FIG. 1.

As shown in FIG. 1, in a configuration according to the embodiment, an amount-of-drinking management system 10 includes a management server 11 and various types of information processing apparatus used by users (a patient and people concerned with the patient) and doctors and the like (as examples of the information processing apparatus, personal digital assistants, smart phones, or tablet terminals 12, 17, and 18; a cell phone 13; and PCs 14 to 16 are shown in FIG. 1. Hereinafter, these apparatus may be collectively referred to as “various types of terminals” or simply as “terminals”). As shown in FIG. 1, the management server 11 is connected with the various types of terminals via private lines or public lines (as wired lines, 37 to 39) such as the Internet so as to be able to communicate with each other. The lines may be either wired or wireless. When the lines are wireless, the personal digital assistants or smart phones 12, 17, and 18 and the cell phone 13 access the Internet 39 wirelessly via not-illustrated base stations or access points and connected with the management server 11 via a line 38 so as to be able to communicate with each other.

Here, the access points are radios used to connect wireless terminals such as PCs or smart phones with each other or with other networks. Typically, radios such as the access points are devices designed to operate under communications protocols on the first layer (physical layer) and the second layer (data link layer) of the ISO Reference Model.

Note that at the time of the filing of the present application, many of cell phones, and personal digital assistants or tablets, which have processing power (communications processing rate, image processing power, or the like) equivalent to that of personal computers (PCs), can be called small computers.

A program or software necessary for the practice of the present invention is normally installed or stored in an HDD, an SSD, or the like in a storage unit of a PC or a personal digital assistant. During execution of the program or software, all or some of software modules are read out into a memory in the storage unit as required and executed on a CPU.

Alternatively, a browser-based computer or a personal digital assistant can be adopted. In that case, the program is delivered to the terminal from another server or a computer as required, and the program is executed by a browser on the terminal.

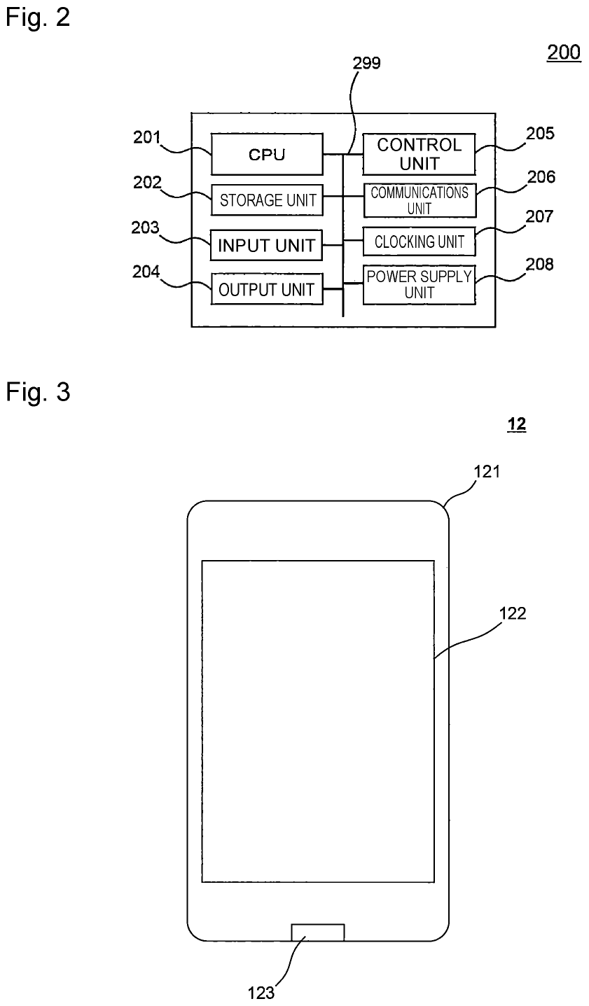

A hardware configuration of the management server 11 can basically adopt a PC as well (this will be described later with reference to FIG. 2 just to make sure). Note that the management server 11 can also adopt a configuration suitable for processing large-scale data by running multiple PCs (as an example, dozens of to tens of thousands of PCs) in parallel to upgrade hardware specifications of the management server 21 as required although the present invention is not limited to this.

A functional block diagram of the management server in the amount-of-drinking management system according to an embodiment of the present invention is shown in FIG. 2. As an example, operation of the management server is implemented by individual operations of hardware described below and coordinated operations of software and the hardware.

In FIG. 2, a management server 200 as an entire hardware block is broadly divided into a CPU 201 that performs various comparison and computation processes; a storage unit 202 including a RAM, a ROM, and a flash memory; an input unit 203 including a keyboard and a pointing device; an output unit 204 including a display and a speaker; a control unit 205 used to control various signals; a communications (interface) unit 206 (regardless of wireless one or wired one); a clocking unit 207 used to count time and the like; and a power supply unit 208.

These modules are connected as required by a communications bus or service wires (the wires are shown together as wire connections 299 with individual wires being divided as appropriate for the sake of convenience in FIG. 2) as appropriate.

The program or software that is necessary for the practice of the present invention and executed on the management server 200 is normally installed or stored in a hard disk, a solid state drive (SSD), or a flash memory making up the storage unit 202. During execution of the program or software, all or some of software modules are read out into memory in the storage unit 202 as required and executed on the CPU 201.

Note that computations do not necessarily have to be executed by a central processing unit such as the CPU 201, and an auxiliary processing unit such as a not-illustrated digital signal processor (DSP) can be used as well.

FIG. 3 shows an external appearance configuration of the smart phone or tablet terminal 12 as an information processing apparatus (user terminal) in the amount-of-drinking management system according to an embodiment of the present invention. In FIG. 3, the smart phone or tablet terminal 12 includes a casing 121, a display 122, and a hardware button 123 provided in a lower center of the casing 121. The display 122 is typically made up of a liquid crystal display (LCD) or the like and capable of displaying various information including characters, still images, and moving images. Menu boutons or a software keyboard may be displayed on the display 122 for the user to give instructions (commands) to the tablet terminal 12 by touching the menu boutons or the software keyboard with fingers or a touch pen (not shown). In this regard, the hardware bouton 123 is not an essential component, but is implemented as a bouton serving a certain function for convenience of explanation of the present invention. According to an embodiment of the present invention, physical boutons such as the hardware bouton 123 can be replaced with menu boutons displayed in part of the display 122.

The display 122 includes a multi-touch input panel and touch position coordinates on the touch input panel are transmitted to a processing system (CPU) of the smart phone or tablet terminal 12 via an input device interface (not shown) and processed there. The multi-touch input panel is configured to be able to simultaneously sense multiple contact points to the panel. The detection (sensor) can be implemented by various methods, and is not necessarily limited to a touch sensor. For example, indicated points to the panel can be extracted using an optical sensor. Furthermore, in addition to contact sensors and optical sensors, capacitive sensors adapted to sense contact with human skin are also available for use.

Although not shown in FIG. 3, the smart phone or tablet terminal 12 can also be equipped with a microphone and a speaker. In that case, the user's voice or the like picked up by the microphone can also be discriminated and used as an input command. Furthermore, although not shown in FIG. 3, a CMOS or other camera device can be mounted on the back or other places of the smart phone or tablet terminal 12.

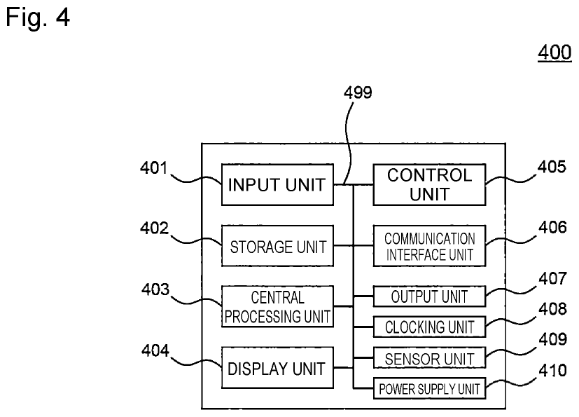

A functional block diagram of hardware making up the smart phone or tablet terminal 12 according to an embodiment of the present invention is shown by example in FIG. 4. Operation of the smart phone or tablet terminal 12 is implemented by individual operations of hardware described below and coordinated operations of software and the hardware.

In FIG. 4, a smart phone or tablet terminal 400 as an entire hardware block is broadly divided into the hardware bouton 123 shown in FIG. 3; the multi-touch input panel provided on the display 122 shown in FIG. 3; an input unit 401 made up of a microphone and the like; a storage unit 402 made up of a hard disk, a RAM, and/or a ROM used to store programs and data; a central processing unit 403 made up of a CPU adapted to perform various numerical calculations and logic operations based on programs; a display unit 404 made up of the display 122 and the like; a control unit 405 used to control chips, an electrical system, and the like; a communications interface unit 406 made up of a slot for use to access the Internet, a port for use to conduct optical communications, and a communications interface; an output unit 407 including a speaker, a vibrator, and an infrared projector; a clocking unit 408 used to count time and the like; a sensor unit 409 made up of a CMOS or other image sensors, an infrared sensor, an inertial sensor, and the like; and a power supply unit 410 used to supply power to individual modules in the apparatus. These modules are connected as required by a communications bus or service wires (the wires are shown together as wire connections 499 with individual wires being divided as appropriate for the sake of convenience in FIG. 4) as appropriate.

Note that the sensor unit 409 may include a GPS sensor module for use to identify the location of the smart phone or tablet terminal 400 (12). Signals detected by a CMOS or other image sensors, an infrared sensor, and the like making up the sensor unit 409 can be processed as input information in the input unit 401.

The program or software that is necessary for the practice of the present invention and executed on the smart phone or tablet terminal 400 is normally installed or stored in a hard disk, a solid state drive (SSD), or a flash memory making up the storage unit 402. During execution of the program or software, all or some of software modules are read out into memory in the storage unit 402 as required and executed on the CPU 403.

Note that computations do not necessarily have to be executed by the central processing unit 403 such as a CPU, and an auxiliary processing unit such as a not-illustrated digital signal processor (DSP) can be used as well.

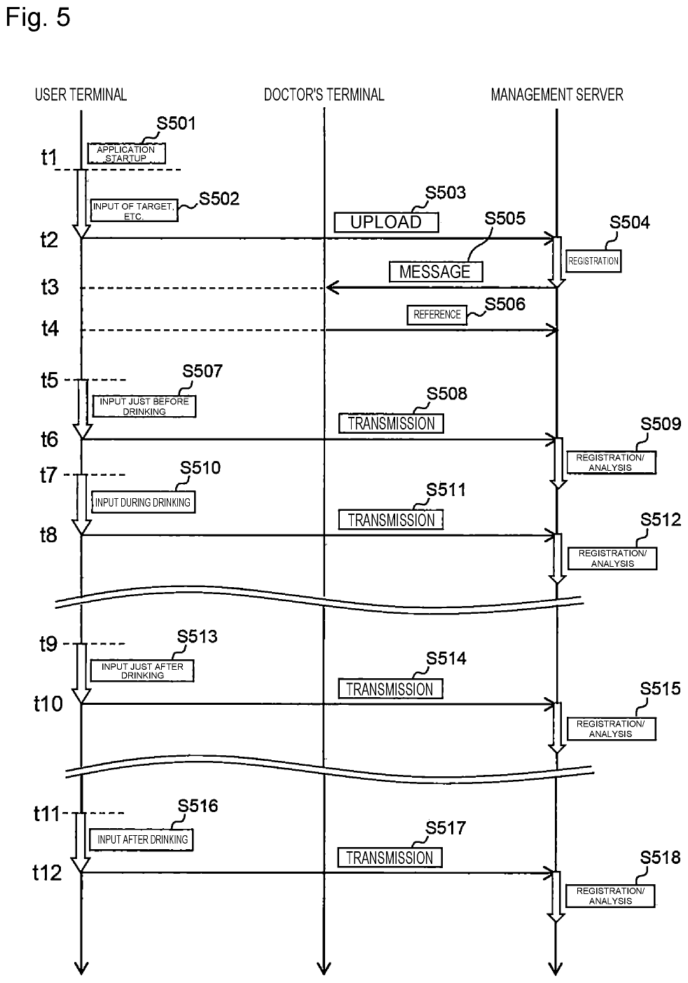

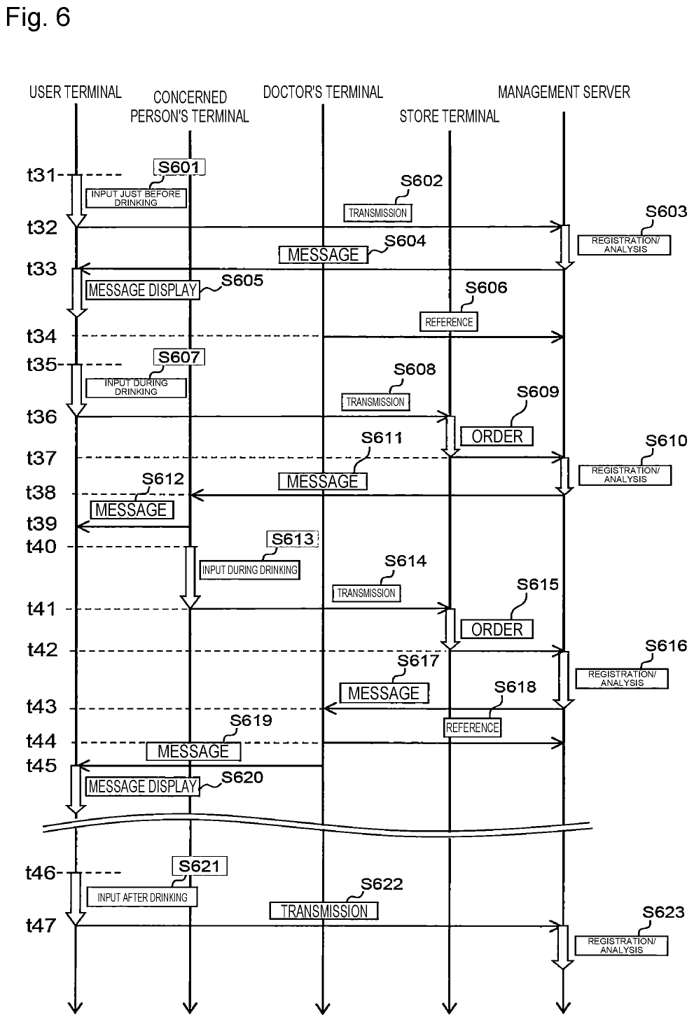

Next, flows of processing operations in the amount-of-drinking management system according to an embodiment of the present invention are shown in FIGS. 5 and 6. The processing operation flow shown in FIG. 5 illustrates by example, along the time axis (time t1 to time t12), how the user starts up an application according to an embodiment of the present invention, enters settings of target values and the like for drinking, and enters inputs before drinking, during drinking, and after drinking, in an embodiment of the present invention. As illustrated, in the meantime, the management system records or registers and manages information entered and transmitted from the user terminal, and analyzes the information as appropriate. According to the embodiment, via a doctor's terminal (a terminal possessed by the user's attending doctor; the same applies hereinafter), the user's attending doctor can also refer, as appropriate, to information managed by the management system. Note that messages not shown in FIG. 5 can be exchanged between the system and the terminals.

FIG. 6, in which a terminal of a person concerned with the user and a store terminal are added to the terminals (user terminal and doctor's terminal) shown in FIG. 5, illustrates an operation flow by example along the time axis (time t31 to time t47) when information is exchanged between the terminals and the management server.

Note that to implement the user's drinking management described with reference to FIGS. 5 and 6, on the amount-of-drinking management system according to an embodiment of the present invention, attribute information (as an example, user attribute information such as user name, full name, contact address, and health care information as well as information about the user's drinking) about each user is managed, recalculated each time, and subjected to update control. The information about the user's drinking is converted into numerical form and recalculated each time, and knowledge information based on calculation results can be transmitted to the user terminal as appropriate.

At time t1 in FIG. 5, the user starts up a dedicated application via his/her own terminal (information processing apparatus) (step S501). Note that it is assumed that necessary processes such as installation have been completed up to this point.

Subsequently, the user can receive desired information notification or receive messages from the doctor and the like via the management server although the present invention is not limited to this.

From time t1 to time t2, the user enters settings of target values for treatment or physical condition improvement of himself/herself (step S502). According to the embodiment, the target values are settings of the amount of drinking, frequency of drinking, and the like to be entered.

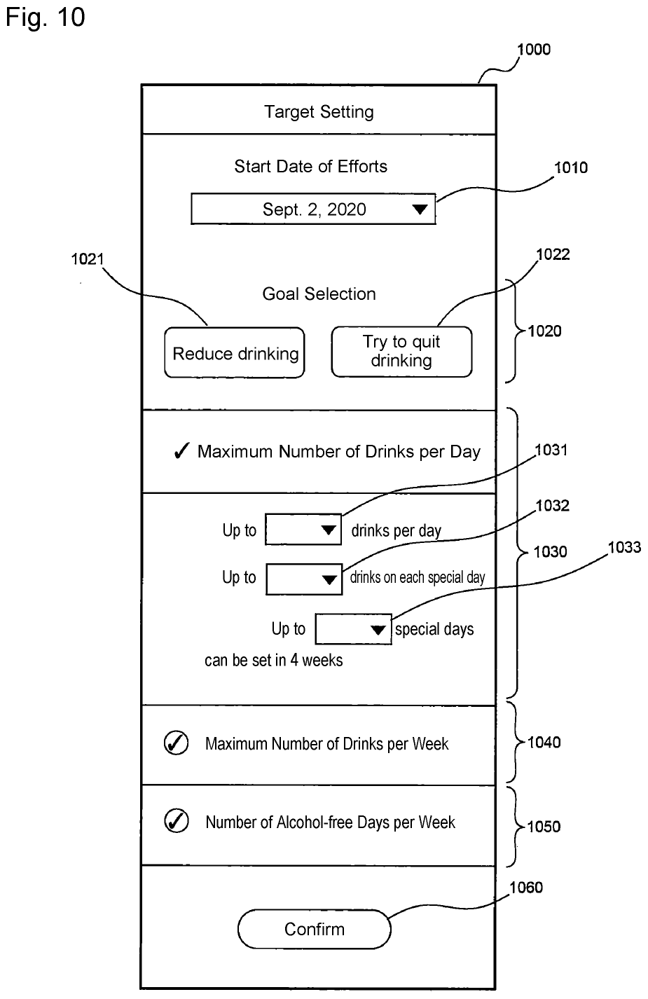

An example of a setting input screen for this is shown in FIG. 10.

FIG. 10 is an exemplary setting input screen used to enter settings of target values for user's drinking. In FIG. 10, a screen 1000 displays a start date of efforts input field 1010, a Goal Selection field 1020, a Maximum Number of Drinks per Day input field 1030, a Maximum Number of Drinks per Week input field 1040, a number of Alcohol-free Days per Week setting field 1050, and a Confirm button 1060. In FIG. 10, the Maximum Number of Drinks per Day input field 1030 is in an open state and more detailed input items 1031 to 1033 can be checked. On the other hand, the Maximum Number of Drinks per Week input field 1040 and the Number of Alcohol-free Days per Week setting field 1050 are in a closed state, and detailed input items are hidden (according to the embodiment, the fields 1040 and 1050 can be in the open state as with 1030 by pressing the check marks on the left end of the respective fields).

According to an embodiment of the present invention, the Goal Selection field 1020 provides two choices: “Reduce drinking” and “Try to quit drinking.” According to the embodiment, when the goal is to reduce drinking, the patient presses a button 1021, and when the goal is to quit drinking, the patient presses a button 1022.

The detailed input items 1031 to 1033 of the Maximum Number of Drinks per Day input field 1030 allow detailed inputs of the amount of drinking, frequency of drinking, and the like. The detailed input item 1031 allows the user to enter the amount of drinking per day in terms of a maximum number of drinks, and according to the embodiment, numerical values can be entered by selecting them from a selection box or the like. Various definitions can be made regarding the number of drinks (drink) in the drinking, and according to the embodiment, 10 g of pure alcohol can be defined as one drink. In this case, 500 ml of beer with an alcohol content of 5% is regarded as “2 drinks” (because the specific gravity of alcohol is 0.8). According to another embodiment, 10 g of pure alcohol can be defined as one drink. In this case, 500 ml of beer with an alcohol content of 5% is regarded as “2.0 drinks.”

According to an embodiment of the present invention, the detailed input item 1032 is designed to allow the amounts of drinking to be set individually for special days (the user's own birthday, wedding anniversary, and the like) determined by the user in advance. Alternatively, according to another embodiment, a special day can be set by the user at any time (e.g., since the user worked hard today, the user hastily designated this day as a special day). Note that normally the number of drinks entered in the input item 1032 is larger in value than the number of drinks entered in the input item 1031, but the present invention is not restricted to this.

Furthermore, the detailed input item 1033 allows the user to set in advance a target value specifying a maximum number of special days that can be set in 4 weeks. According to the embodiment, the user can set desired days in 4 weeks as special days as long as the special days do not exceed the target value specified here. Furthermore, according to another embodiment, the user can set a special day after the event.

The period of “4 weeks” in input item 1033 may be replaced by any of various other periods such as 3 months, half a year, and one year.

The Maximum Number of Drinks per Week input field 1040 allows the user to specify an upper limit to the number of drinks the user permits himself/herself to take each week. The Number of Alcohol-free Days per Week setting field 1050 allows the user to specify the number of alcohol-free days in one week. Here, the period of “one week” may be replaced by any of various other periods such as 2 weeks and one month. Regarding the alcohol-free day setting, not simply the number of days, but also one or more days of week can be specified (as an example, a certain day every week or multiple days of week such as the first and third Sundays and Mondays).

Once the target setting values are entered, by pressing the Confirm button 1060, the user can upload the input information to the management server (step S503), causing the management server to record, or register and manage, the input information (step S504). According to an alternative embodiment, rather than uploading the input information to the management server with the press of the Confirm button 1060, the input information may be uploaded to the management server with other timing (such as timing when the application goes online).

According to the embodiment, a message is transmitted from the management server to the doctor's terminal at time t3 (step S505) although the present invention is not restricted to this. Various transmission reasons for the message transmission is conceivable. The message may simply be a notice that user registration has been completed or may be a notice encouraging the user to modify the target setting value if the user's target setting value is too high (or too low). According to the embodiment, these conditions can be set in advance on an application installed on the user terminal. The message transmission to the user can also be done even when the user terminal is offline.

At time t3, the doctor's terminal receives the message transmitted from the management server. Here, the doctor checks content of the message received by the doctor's terminal and refers to information registered in the management server to check content of the user's registration (time t4, step S506).

Next, from time t5 to time t6, the user enters inputs just before starting drinking (step S507). In this case, various input items can be adopted, but according to the embodiment, the input items shown in Table 1 are adopted.

TABLE 1

| | | | | |
|---|---|---|---|---|
| With | Where? | With what | When do you | Under what |
| whom? | | feeling? | start drinking? | circumstances? |

Of the items in Table 1, regarding the item “With whom?,” the user may enter a specific name or may enter only a relationship (family, friend, or the like) with the user. Alternatively, the user may enter simply the number of persons. Furthermore, these input forms may be used in combination rather than being unified.

Regarding the item “Where?,” the user may enter a specific place name (name or address of the drinking place) or may enter a broad category (own house, friend's house, or drinking place).

Regarding the item “With what feeling,” typically the user may select from predetermined categories (“pleasant,” “bitter,” “angry (feeling of anger),” etc.).

Regarding the item “When do you start drinking?,” the user may enter the date and time in advance if the date and time are decided.

Regarding the item “Under what circumstances?,” according to the embodiment, the user may enter a situation concerned with the user himself/herself who is going to drink, such as “class reunion,” “entertainment,” or “birthday party for myself or someone else.”

In FIG. 5, when the inputs just before drinking are finished, the entered data is transmitted to the management server (step S508) and the transmitted information is recorded or registered in the management server (step S509). The management server not only can record or register the information, but also can aggregate and analyze the information as required (step S509; various types of aggregation and analysis will be described later). Although not illustrated thoroughly in FIG. 10, aggregation and analysis results produced by the management server can be fed back to the user as appropriate (various types of feedback will be described later).

Next, from time t7 to time t8, the user enters inputs during drinking (step S510). In this case, various input items can be adopted, but according to the embodiment, the input items shown in Table 2 are adopted.

TABLE 2

| | | | | |
|---|---|---|---|---|
| When? | What? | How much? | With what | Under what |
| | | | feeling? | circumstances? |

Of the items in Table 2, regarding the item “When?,” the time (date and time) at the time of entering inputs may be entered automatically. Regarding the item “What?,” the user may enter the type of alcoholic drink (alcohol content and amount) the user is drinking. Regarding the item “With what feeling,” the user may enter changes in feeling. That is, the user may enter not only simply “pleasant,” but also the degree of pleasantness in numerical form.

Regarding the item “Under what circumstances?,” the user may enter changes in the gathering (e.g., there was a walk-in participant and the gathering was boosted further, or conversely, a sad topic was brought up and the gathering suddenly became depressed).

In FIG. 5, when the inputs during drinking are finished, the entered data is transmitted to the management server (step S511) and the transmitted information is recorded or registered in the management server (step S512). The inputs during drinking may be entered multiple times during drinking. The management server not only can record or register the information, but also can aggregate or analyze the information as required (step S512; various types of aggregation and analysis will be described later). In step S512, by comparing with content of the inputs during drinking entered the previous time (e.g., 15 minutes ago) the management server can record the order in which the user took alcoholic drinks in the place or calculate a drinking pace based on drink-by-drink input intervals of a drinking record.

Although not illustrated sequentially in FIG. 10, aggregation and analysis results produced by the management server can be fed back to the user as appropriate (various types of feedback will be described later).

Next, from time t9 to time t10, the user enters inputs just after drinking (step S513). For the input items in this case, various input items can be adopted, but according to the embodiment, the input items shown in Table 3 are adopted.

TABLE 3

| | |
|---|---|
| When? | With what feeling? |

Of the items in Table 3, regarding the item “When?,” the time (date and time) at the time of entering the inputs may be entered automatically. Regarding the item “With what feeling,” the user may also enter changes in feeling or the degree of satisfaction.

In FIG. 5, when the inputs just after drinking are finished, the entered data is transmitted to the management server (step S514) and the transmitted information is recorded or registered in the management server (step S515). The management server not only can record or register the information, but also can aggregate or analyze the information as required (step S515; various types of aggregation and analysis will be described later).

Although not illustrated sequentially in FIG. 10, aggregation and analysis results produced by the management server can be fed back to the user as appropriate (various types of feedback will be described later).

Next, from time t11 to time t12, the user enters inputs after drinking (step S516). According to an embodiment of the present invention, the inputs are entered before bedtime on the date of drinking or the next morning. For the input items in this case, various input items can be adopted, but according to the embodiment, the input items shown in Table 4 are adopted.

TABLE 4

| | | |
|---|---|---|
| Physical condition | Feeling | Sleep |

Of the items in Table 4, regarding the item “Physical condition,” if the input is entered on the day of drinking, the physical condition of the user at application logout time is entered, and if the input is entered on the day after drinking (e.g., the next morning), the physical condition of the user at application login time is entered. In so doing, the time (date and time) at the time of entering the inputs may be entered automatically. Regarding the item “Feeling,” the user may enter the state of feeling after the drinking.

In FIG. 5, when the inputs after drinking are finished, the entered data is transmitted to the management server (step S517) and the transmitted information is recorded or registered in the management server (step S518). The management server not only can record or register the information, but also can aggregate and analyze the information as required (step S518; various types of aggregation and analysis will be described later).

Although not illustrated sequentially in FIG. 10, aggregation and analysis results produced by the management server can be fed back to the user as appropriate (various types of feedback will be described later).

The flow shown in FIG. 6 is a variation of the flow shown in FIG. 5, and includes a concerned person's terminals and a store terminal. According to the embodiment, the concerned person's terminal is a terminal possessed by a person concerned with the user (family member, friend, or the like) while the store terminal is a terminal installed in a barroom visited by the user and the like for drinking and the like.

Each of the concerned person's terminal and the store terminal in FIG. 6 corresponds to any of the various types of terminals 12 to 18 in FIG. 1. In FIG. 6, it is assumed that installation of a drinking management application and setting of a target value on the user terminal and the like have already been completed.

Next, from time t31 to time t32 in FIG. 6, the user enters inputs just before starting drinking (step S601). For the input items in this case, various input items can be adopted, but according to the embodiment, the input items such as “With whom?,” “Where?,” “With what feeling?,” “When do you start drinking?,” and “Under what circumstances?” are adopted as already described above.

In FIG. 6, when the inputs just before drinking are finished, the entered data is transmitted to the management server (step S602) and the transmitted information is recorded or registered in the management server (step S603). The management server not only can record or register the information, but also can aggregate or analyze the information as required (step S603). Aggregation and analysis results produced by the management server can be fed back to the user as appropriate (step S604).

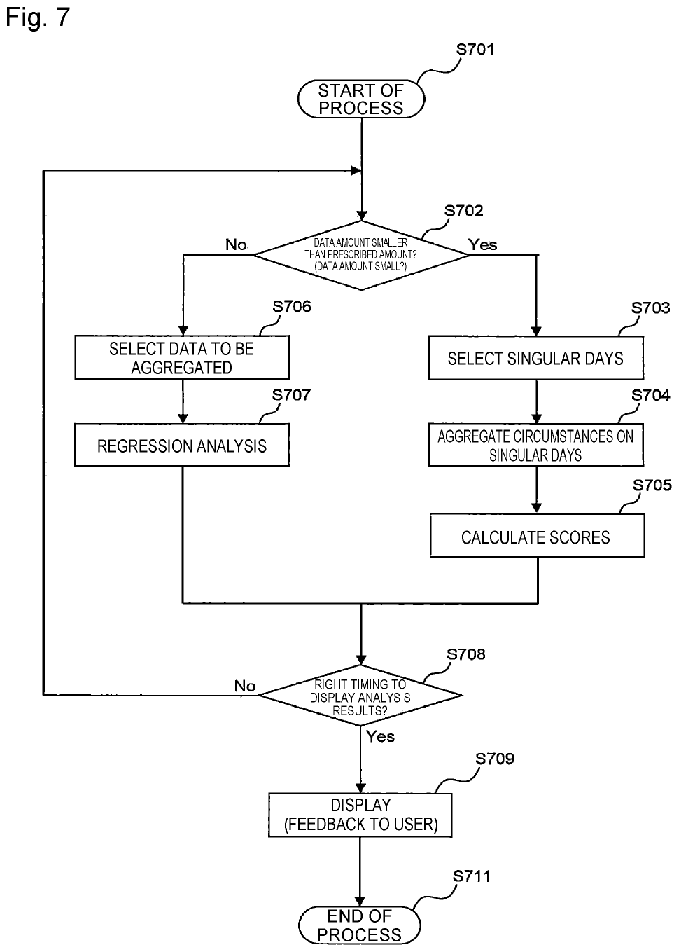

An exemplary flow of an analysis process performed by the management server in step S603 is shown in FIG. 7. Note that the process flow shown in FIG. 7 is carried out not only during the input just before drinking such as in step S603, but also with any timing without causing any technical contradiction in an embodiment of the present invention. In the exemplary analysis process flow shown in FIG. 7, as an example, control is performed such that the process will be divided according to whether an amount of data (the number itself of subjects or an amount of data acquired from a specific subject) already managed by the management server is small or large. According to the embodiment, if the amount of data is small, an analysis process shown in steps S703 to S705 is performed, and if the amount of data is large, an analysis process shown in steps S706 and S707 is performed. In this case, to determine whether the amount of data is small or large, a predetermined threshold may be adopted. Different thresholds may be used in determining whether the amount of data is small and whether the amount of data is large (in this case, when it cannot be said that the amount of data is either small or large, either another publicly known analysis process or a process according to a variation described in the present embodiment is performed).

The exemplary analysis process flow will be described in detail below with reference to FIG. 7.

When processing starts in step S701 in FIG. 7, the system goes to step S702 and the management server determines whether the amount of data already recorded or registered and managed is small or large. As described earlier, a data amount parameter may be the number of subjects (users) that can be compared or quantity of data acquired from each subject (user). If it is determined in step S702 that the amount of data is small (Yes), the system goes to step S703, but if it is determined in step S702 that the amount of data is large (No), the system goes to step S706.

In steps S703 to S705, some “singular days” are set and other circumstances on the singular days are aggregated and scored. According to an embodiment of the present invention, days such as the following are set as “singular days.”
(A1) The day's total amount of drinking is a record high.
(A2) The day's total amount of drinking is in the top xx % of the past drinking days.
(A3) The day's amount of drinking has exceeded a heavy drinking standard.
(A4) The day's amount of drinking has exceeded a target setting value.

According to an embodiment of the present invention, by considering various circumstances (as an example, “With whom?,” “Where?,” “With what feeling?,” “When did you start drinking?,” and “Under what circumstances?”) on the singular days, the singular days under each circumstance are aggregated.

Regarding scoring, as an example, scores are calculated as follows, and based on the scores, input items in each category (e.g., “Where?”) can be ranked. Here, the input item is, for example, “Home” in response to “Where?”.
(B1) The proportion (%) in which the input item is contained in the top x % of the amount of drinking days/(the proportion (%) in which the input item is contained in drinking days other than the top x % of the amount of drinking days+1)
(B2) The proportion (%) in which the input item is contained in the days exceeding the heavy drinking standard/(the proportion (%) in which the input item is contained in the drinking days not reaching the heavy drinking standard+1)
(B3) The proportion (%) in which the input item is contained in the days exceeding an upper limit of a target amount of drinking/(the proportion (%) in which the input item is contained in the days exceeding the upper limit of the target amount of drinking+1)
(B4) The average degree of dissatisfaction among the days containing the input item (satisfied=−1, indifferent=0, dissatisfied=1)
Regarding the expression (B1) described above, when the expression is solved, if drinking at “home” is highly relevant to the top x % of the amount of drinking days, the numerator increases, and so does the value of the score. If the relevancy is low, the denominator increases and the value of the score decreases. Furthermore, to keep the denominator from becoming 0, “+1” is added (this is also true for the expressions (B2) to (B4) described above).

Returning to FIG. 7, singular days are selected according to circumstances in step S703. Regarding a method for the selection, the selection may be made in a fixed manner in advance or singular days may be selected in a predetermined order. Alternatively, to aggregate circumstances on all singular days, all the singular days may always be selected.

Next, the system goes to step S704, and performs aggregation under each circumstance on each singular day. This makes it possible to prepare data for use to understand under what circumstance drinking tends to be prominent when attention is paid to the given singular day. Then, the system goes to step S705 and calculates scores and the like by paying attention to each of the singular days.

Note that it is not essential to calculate indices in both steps S704 and S705, and the process in one of the steps may be omitted depending on the state of implementation. In that case, feedback is provided to the user based on either aggregation results concerning circumstances on the singular days (step S704) or results of scoring (step S705).

On the other hand, in steps S706 and S707, because the management server possesses abundant data to be processed, after data to be aggregated is selected (step S706), a determination is made based on publicly known regression analysis. As the publicly known regression analysis, linear regression analysis, logistic regression analysis, or the like is adopted. Furthermore, publicly known decision tree analysis may be used.

In step S706, to select the data to be aggregated, a selection is made as to whether the data is accumulated data of the user himself/herself or data per unit of audience (demographic attribute).

If regression analysis is adopted in step S707, calculations and the like are performed based on the following general formula.

$$Y = a 1 \times 1 + a 2 \times 2 + a 3 \times 3 + … + error term$$

According to an embodiment of the present invention, as term Y in the above formula, “the day's total amount of drinking,” “degree of satisfaction,” “values of physical condition, feeling, sleepiness, and the like at logout on the day,” “values of physical condition, feeling, degree of sound sleep, and the like at login on the next day,” and the like can be adopted. As term X in the above formula, “values of physical condition, feeling, sleepiness, and the like at login on the day,” and values of “with whom, where, with what feeling,” and the like can be adopted. Values of “when did you start drinking (time),” “when did you start drinking (circumstance),” “type of alcoholic drink you took (alcohol content and amount),” “the order in which you took alcoholic drinks,” and “drinking pace (calculated from the duration of input on the drinking record),” can also be adopted as term X in the above formula.

In the above formula, terms a1, a2, a3, . . . are regression coefficients.

In step S708 in FIG. 7, it is determined whether timing is right to display analysis results for the user. According to an embodiment of the present invention, the following timings are used in step S708.
(C1) When requested by the user.
(C2) Timing set by the application except during recording of the user's drinking.
(C3) When a score or the like exceeding a threshold is detected during recording of drinking.

Specific scene examples of (C1) include a case in which the user makes an inquiry to the management server when the user wants to know in what cases the user's amount of drinking tends to increase. In this case, the management server conducts the analysis described above based on a past record and feeds back results to the user. Specific scene examples of (C3) include a case in which the management server conducts analysis at any time during the input during drinking and transmits a message to the user to alert the user if a predetermined threshold is reached.

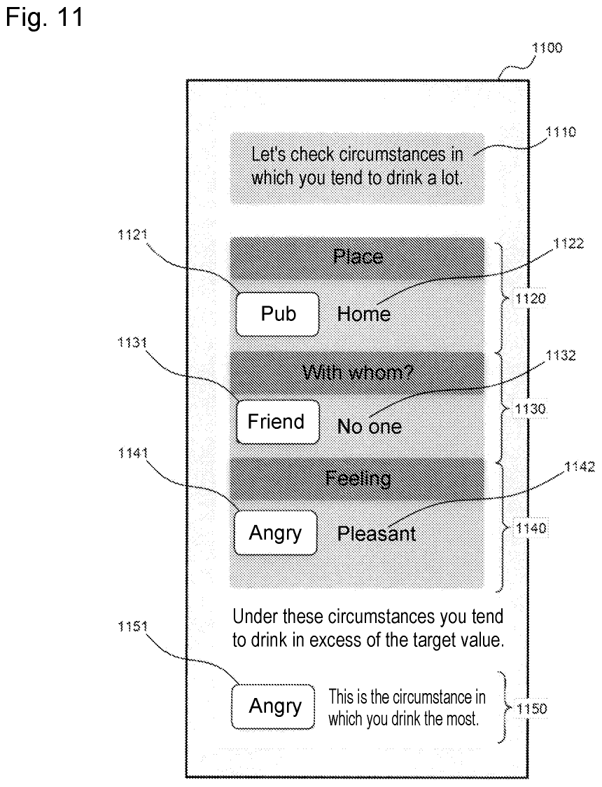

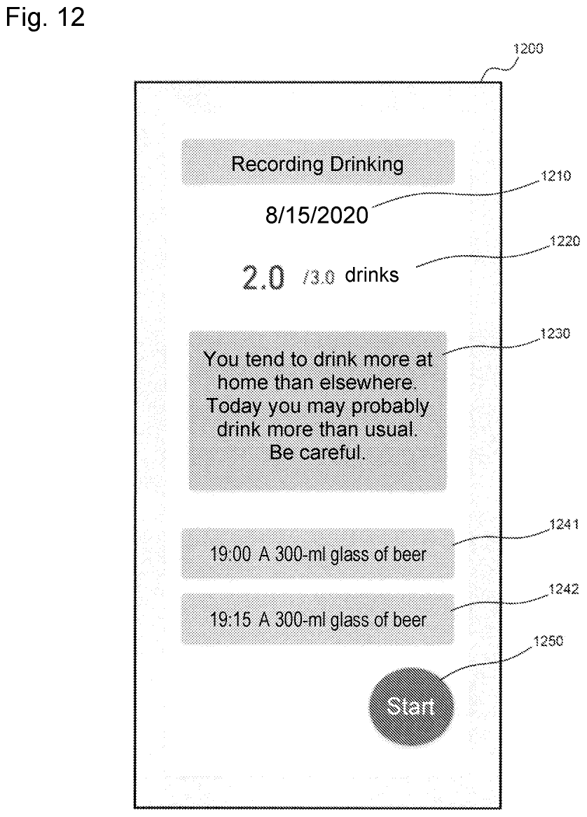

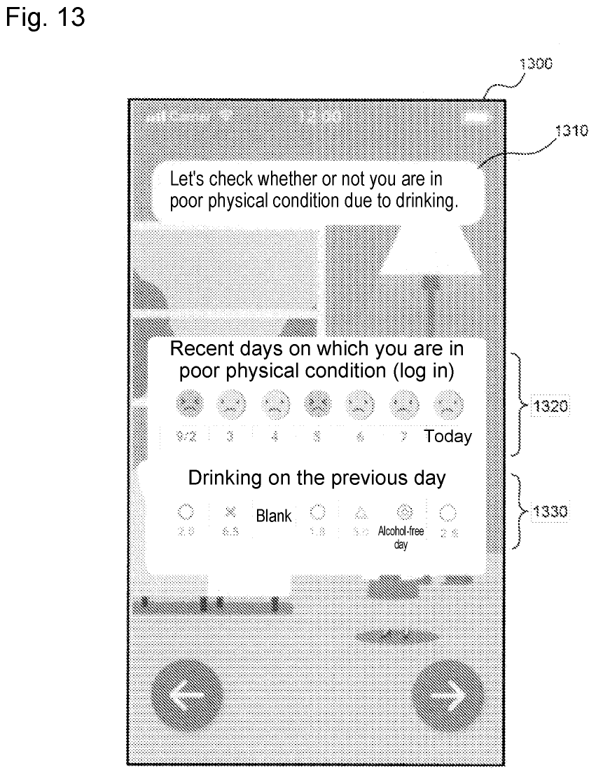

Returning to FIG. 6 again, in step S605, the feedback from the management server is displayed on the user terminal (step S605). In an embodiment of the present invention, FIGS. 11 to 13 are shown as examples of a feedback screen. In step S605, a screen like FIG. 10 is displayed to present objective information before the user's drinking to make it easy for the user to drink, or a screen like FIG. 13 is displayed to suggest a relationship between the user's drinking and physical condition based on analysis of the user's past drinking history. Alternatively, although not illustrated in FIG. 6, a screen like FIG. 12 may be displayed on the user terminal to alert the user if the user's undesirable drinking tendency is found while the user is entering inputs during the user's drinking as in step S607 described later.

Next, at time t34 in FIG. 6, the doctor accesses the management server via the doctor's terminal to refer to user's information (step S606).

At time t35 in FIG. 6, the user enters inputs during drinking via the user terminal. Here, according to an embodiment of the present invention, input information is transmitted to a store terminal installed in the eating/drinking house (such as a steakhouse or a pub) being visited by the user instead of being transmitted directly to the management server (time t36; step S608). That is, the input during drinking in step S607 is the drinking record of the user and at the same time is a drink order in the store. In other words, the user's drink order in the store also may serve as a record entered by the user himself/herself during drinking. This is possible because at the time of the filing of the present application, the technological level is such that orders can be placed not only from a terminal installed in the store, but also from an application installed on the terminal of the user himself/herself. In this case, the system and the application can be linked together as follows, as required.
(D1) The input during drinking in step S607 is entered to an amount-of-drinking management application, and orders are placed to the store terminal from the amount-of-drinking management application.
(D2) The input during drinking in step S607 is entered as a drink order in the store to a store application distributed by the eating/drinking house, and the store application and the amount-of-drinking management application and/or the management server are linked together as appropriate (not shown in FIG. 6).
(D3) The input during drinking in step S607 is entered to the amount-of-drinking management application and the entered data is transmitted to, and registered in, the management server once, and then a drink order is placed to the store terminal via the management server (not shown in FIG. 6).

From time t36 to time t37 in FIG. 6, order processing is performed on the store terminal (step S609). At time t37, information on the input during drinking is transmitted from the store terminal to the management server. The information is recorded or registered, and aggregated and analyzed by the management server (step S610).

When the management server performs aggregation and analysis in step S610, if an analysis result exceeds some threshold and consequently it is determined that feedback should be provided to the user, for example, a message 1230 as shown in FIG. 12 may be transmitted directly to the user terminal, but in FIG. 6, as another embodiment, a message is transmitted to a terminal (the concerned person's terminal) possessed by a person concerned with the user (family member, friend, or the like) (time t38; step S611). Content of the message in step S611, for example, is a message that asks the person concerned to encourage the user to restrict his/her drinking (the content of the message is not shown). The person concerned who receives the message in step S611, understands its content and transmits a message via the person's terminal, calling the user for restraint from drinking (time t39; step S612). The messages in step S612 may be exchanged via the amount-of-drinking management application or transmitted via another messaging application or the like (in this case, it can be said that a mechanism of message transmission is implemented under the control of the system according to an embodiment of the present invention).

Next, from time t40 to time t41, the person concerned who is sitting with the user is entering inputs during drinking for the user using the concerned person's terminal (step S613). Contents of the input items are the same as those described already above. According to an embodiment of the present invention, the people concerned with the user can support the user in treatment and physical condition improvement of the user in this way by installing the amount-of-drinking management application. The entered information is transmitted from the concerned person's terminal to the store terminal (step S614), and the store terminal processes the information as an order (time t41 to time t42; step S615). The information on the input during drinking is transmitted to the management server (time t42). Note that whereas in steps S614 and S615, the user's inputs during drinking are also processed as a store order, the present invention is not limited to this, and the user's inputs during drinking may be transmitted directly to the management server without involving the store terminal.

The received information on the input during drinking is recorded or registered, and aggregated and analyzed by the management server (time t42 to time t43; step S616).

At time t43 in FIG. 6, a message is transmitted from the management server to the doctor's terminal (time t43; step S617). This is because matters determined to be preferably fed back to the user have been extracted as a result of the aggregation and analysis. According to another embodiment of the present invention, the message can be transmitted directly to the user terminal without involving the doctor's terminal.

According to an embodiment of the present invention, in step S617, a message is transmitted to the user from the attending doctor, encouraging the user to restrain himself/herself from drinking (the content of the message is not shown). At time t44, the doctor accesses the management server via the doctor's terminal to check drinking status of the user (step S618). Then, at time t45, the doctor transmits a message to the user via the doctor's terminal, calling the user for restraint from drinking (step S619).

The message transmitted from the doctor's terminal is displayed on the user terminal (time t45; step S620).

Although not illustrated in FIG. 6, the user can enter inputs just after drinking (which is different from inputs after drinking described later in step S621).

Next, from time t46 to time t47, the user enters inputs after drinking (step S621). According to an embodiment of the present invention, the inputs are entered before bedtime on the date of drinking or the next morning. When the inputs after drinking are finished, the entered data is transmitted to the management server (step S622) and the transmitted information is recorded or registered in the management server (step S623). The management server not only can record or register the information, but also can aggregate and analyze the information as required (step S623).

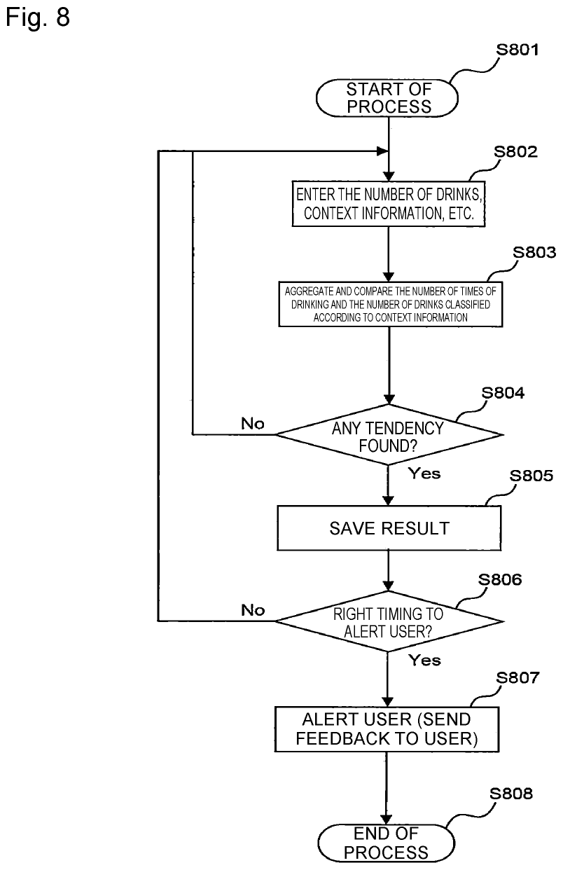

A processing operation flow in the amount-of-drinking management system according to an embodiment of the present invention is shown in FIG. 8. The processing operation flow shown in FIG. 8 is an alternative to the processing operation flow shown in FIG. 7, and is a specific example of analysis process performed by the management server as appropriate when inputs during drinking are entered by the user. The processing operation flow shown in FIG. 8 can be used together with part or all of the processing operation flow shown in FIG. 7 as long as there is no logical contradiction.

Operation will be described in detail below with reference to FIG. 8.

When processing starts in step S801, the system goes to step S802 and allows the user to enter the number of drinks and context information. Information about drinks, which is not limited to the number of drinks, and can include kinds of drink, alcohol content, and quantities, can be entered. The context information is the user's circumstantial information described above (subjective information such as “with what feeling?” as well as circumstantial information such as “with whom?,” “where?,” and “under what circumstances?”). The entered information is transmitted to the management server and is recorded or registered.

Next, the system goes to step S803, and aggregates and analyzes the data recorded or registered in the management server. Here, according to the embodiment, the number of times of drinking and the number of drinks in the drinking are aggregated and compared for each item of the recorded or registered context information (process concepts and the like will be described later).

Next, the system goes to step S804, and determines whether any tendency or matter to be fed back to the user has been found as a result of the aggregation and analysis in the previous step. If the determination is Yes in step S804, the system goes to step S805, but if the determination is No, the system returns to step S802.

In step S805, the result found in the previous step is saved once, and it is determined in step S806 whether the timing is right to alert (provide feedback to) the user. If the determination is Yes in step S806, the system goes to step S807, but if the determination is No, the system returns to step S802.

In step S807, the user is alerted (receives feedback). Specific feedback screens are as shown in FIGS. 11 to 13 (configurations and the like of the respective figures will be described later). Then, the system goes to step S808, and finishes the processing of the flow.

The analysis process in steps S803 and S804 will be described in detail below along the following items: (Basic concept of process), (Specific examples of process), and (Examples of other processes).

### (Basic Concept of Process)

The analysis process according to an embodiment of the present invention is performed based on the amount of drinking (the number of drinks) of the user and context information (“feeling,” “place,” and the like) on the user. As an example, the number of times of drinking and the number of drinks are counted and compared for each item of context information. If the amount of drinking of the user is significantly larger when the feeling is “angry” than when the feeling is “relaxed” or “calm,” and if it is recognized that the amount of drinking is significantly larger when the place of drinking is “tavern” than when the place of drinking is “home,” the management server provides feedback to the user, informing the user that the amount of drinking increases when the feeling of the user is “angry” and the place of drinking is “tavern.”

The management server may perform control such that feedback will be provided before or during drinking when the feeling of the user is “angry” or when the place is “tavern.” Alternatively, the management server may perform control such that feedback will be provided in normal times when the user makes an inquiry to the management server. Besides, the management server may perform control such that feedback will be provided with any timing specified on the amount-of-drinking management application.

### (Specific Examples of Process)

Next, specific examples of the process based on the above basic concept will be shown. Table 5 is a record showing how much the user drank by the day, where, and with what feeling.

TABLE 5

| Date | The number of drinks | Place | Feeling |
|---|---|---|---|
| Sep. 2, 2020 | 2 | Home | Angry |
| Sep. 2, 2020 | 2 | Home | Angry |
| Sep. 2, 2020 | 2 | Home | Angry |
| Sep. 2, 2020 | 2 | Home | Angry |
| Sep. 3, 2020 | 2 | Home | Relaxed |
| Sep. 4, 2020 | 2 | Tavern | Relaxed |
| Sep. 4, 2020 | 2 | Tavern | Relaxed |
| Sep. 4, 2020 | 2 | Tavern | Relaxed |
| Sep. 4, 2020 | 2 | Tavern | Relaxed |
| Sep. 4, 2020 | 2 | Home | Calm |
| Sep. 4, 2020 | 2 | Home | Calm |

Although in Table 5, attention is paid only to the date, the present invention is not limited to this, and parameters such as the time of day and the day of week can also be introduced. Regarding the number of drinks, instead of simply counting the number of glasses, alcohol consumption can be counted in terms of alcohol content or pure alcohol. For ease of understanding the invention, detailed description will be omitted below.

In Table 5, when “total number of drinks,” “total number of days of appearance,” and “total number of drinks/total number of days of appearance” for each context are aggregated or calculated in relation to each context parameter such as “feeling” or “place,” results are as shown in Table 6 below.

TABLE 6

| | | | Total number of |
|---|---|---|---|
| | Total | Total number | drinks/total |
| Context | number | of days of | number of days |
| parameter | of drinks | appearance | of appearance |
| Angry | 8.0 | 1.0 | 8.0 |
| Relaxed | 10.0 | 2.0 | 5.0 |
| Calm | 4.0 | 1.0 | 4.0 |
| Home | 14.0 | 3.0 | 4.7 |
| Tavern | 8.0 | 1.0 | 8.0 |

As a result of the aggregation or calculation of Table 6, it can be seen that the number of drinks increases significantly when the feeling of the user is “angry” or the place of drinking is “tavern.” Based on the results, the management server can provide feedback to the user. The analysis results can be used to propose measures based on the doctor's opinion or the like.

### (Examples of Other Processes)

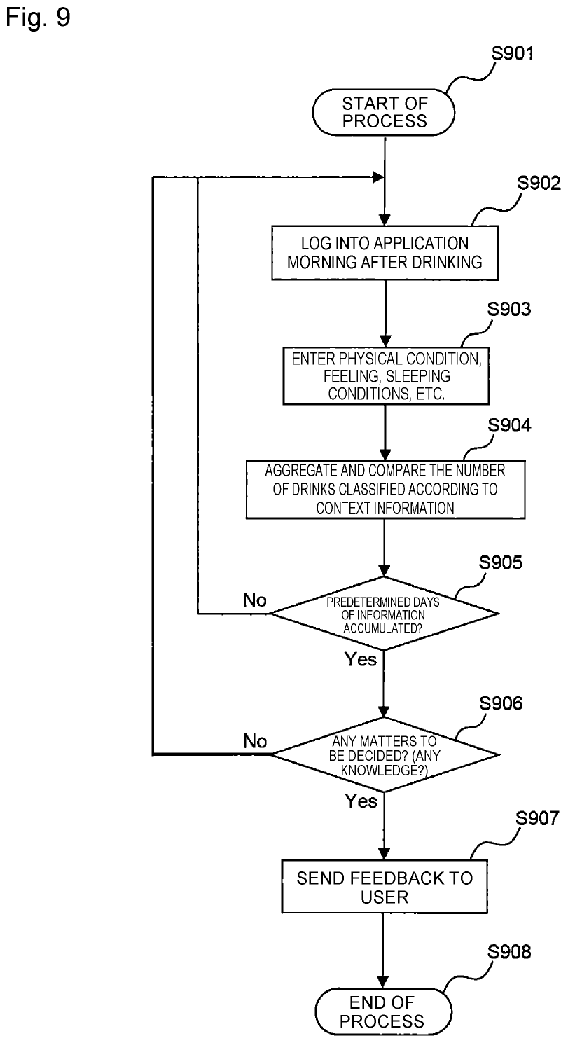

Another processing operation flow in the amount-of-drinking management system according to an embodiment of the present invention is shown in FIG. 9. Whereas the process flow shown in FIG. 8 is an analysis process based on information entered mainly during drinking, the process flow shown in FIG. 9 is an analysis process based on information entered mainly after drinking. However, the flows shown in FIGS. 8 and 9 should not be distinguished exclusively as being completely independent of each other. The flows shown in FIGS. 8 and 9 have been organized for ease of understanding the invention.

Description will be given below with reference to FIG. 9.

When processing starts in step S901, the system goes to step S902 and the user logs into the amount-of-drinking management application in the morning after the drinking day or the like. Next, the system goes to step S903, and the user enters his/her own physical condition and feeling (mood) as well as sleeping conditions of the last night and the like to the amount-of-drinking management application. As an example, these input items can be provided in such a way as to allow selection from multiple choices or in such a way as to allow evaluation on a multi-point scale.

In step S904, on the management server of the amount-of-drinking management system according to an embodiment of the present invention, the number of drinks taken by the user is aggregated for each item of the context entered by the user, and compared and analyzed as required. According to the embodiment, the aggregation, comparison, and analysis can be performed each time the user enters an input and results thereof can be updated as required.

In step S905, an accumulated amount of information entered by the user is determined. According to an embodiment of the present invention, it is determined whether predetermined days of information entered by the user is accumulated. This is because if the accumulated amount of data is small, the meaning of feedback often becomes less significant. Five or seven days are adopted for the predetermined days here as an example, but the present invention is not restricted to this. If the determination is Yes in step S905, the system goes to step S906, but if the determination is No, the system returns to step S902.

Next, the system goes to step S906, and determines whether the results of comparison and analysis performed up to step S904 contain matters to be decided (whether knowledge has been found). Typically, matters exceeding a threshold can be designated as matters to be decided and higher-ranking facts as a result of comparison can be designated as findings. If the determination is Yes in step S906, the system goes to step S907, but if the determination is No, the system returns to step S902.

Note that according to another embodiment of the present invention, the determination process in step S905 may be omitted. If not omitted, the determination process in step S905 may be performed by being interchanged with the determination process in step S906.

In step S907, the matters to be decided and knowledge obtained up to the previous step is fed back to the user. Then, the system goes to step S908, and finishes the flow.

### [Embodiment Focusing on Usage Scenes]

Next, description will be given of an embodiment that focuses on usage scenes on the amount-of-drinking management system according to an embodiment of the present invention. Here, description will be given in a scenario form, illustrating what action the user takes, how the user uses the system, and what feedback the user receives. Here, basically scenes (scenes 1 to 9) proceed in a time series.

### (Scene 1): Target Value Setting

User A (user ID: D001) entered, in advance, targeted upper limit on the amount of drinking in a day, upper limit on the total amount of drinking in a week, and the number of alcohol-free days in a week, to the amount-of-drinking management application. Specifically, when 10 g of pure alcohol was taken to be one drink, the user set 14 drinks as the upper limit on the total amount of drinking in a week.

More specific target setting input data is as shown in Table 7.

TABLE 7

| | | Upper limit on total amount of |
|---|---|---|
| | | drinking in a week (when converted |
| Item ID | User ID | into the number of drinks) |
| 1 | D001 | 3.0 |

### (Scene 2): Input Just Before Drinking and Input During Drinking

From the evening of the day on which the amount-of-drinking management application was installed, user A was going to have a meal that involved drinking. At 19:00, user A entered inputs just before drinking, entering information about the meal involving drinking: “with a friend (whom user A hadn't seen for a long time),” “in a pub,” “in a pleasant mood.” Then, user A started the meal.

With each succeeding drink from the first drink to third drink, user A entered inputs during drinking, entering the number of drinks and context information (“drink,” “with whom,” “place,” and “feeling”) as shown in Table 8 below. Note that date and time are entered automatically at the time of entering the inputs.

TABLE 8

| | | | Type of | Quantity | | | |
|---|---|---|---|---|---|---|---|
| Item | | | alcoholic | in terms | With | Where | |
| ID | Date | Time | drink | of drinks | whom | (place) | Mood |
| 1 | 2021 May 10 | 19:10 | Beer (5%) | 2.0 | Friend | Pub | Pleasant |
| | | | 500 ml | | | | |
| 2 | 2021 May 10 | 19:20 | Beer (5%) | 2.0 | Friend | Pub | Pleasant |
| | | | 500 ml | | | | |
| 3 | 2021 May 10 | 20:30 | Beer (5%) | 2.0 | Friend | Pub | Pleasant |
| | | | 500 ml | | | | |

(Scene 3): Input Just after Drinking

After the meal, user A entered a degree of satisfaction of 4 (on a scale of 5). Subsequently, after parting from the friend, instead of going straight back home, user A stopped in at a bar alone for drinking (entered inputs during drinking anew, subsequently entering 2 as a degree of satisfaction (on a scale of 5)).

(Scene 4): Input after Drinking

User A returned home that day, took a shower, and went to bed. The next morning user A started up the amount-of-drinking management application. After login, user A entered his/her own physical condition, feeling, sleep information, and the like. As an example, user A entered 3 (on a scale of 5) for physical condition, 2 (on a scale of 5) for feeling, and 2 (on a scale of 5) for sleep.

### (Scene 5): After a Lapse of Seven Days

Using the amount-of-drinking management application in the manner described above, user A entered, as an example, seven days of data, such as shown in Table 9, as a drinking record (there is no entries on the 2nd, 5th, and 7th days, which were alcohol-free days).

TABLE 9

| | | | Type of | Quantity | | | |
|---|---|---|---|---|---|---|---|
| Item | | | alcoholic | in terms | With | Where? | |
| ID | Date | Time | drink | of drinks | whom? | (place) | Mood |
| 1 | 2021 May 10 | 19:10 | Beer (5%) | 2.0 | Friend | Pub | Pleasant |
| | | | 500 ml | | | | |
| 2 | 2021 May 10 | 19:20 | Beer (5%) | 2.0 | Friend | Pub | Pleasant |
| | | | 500 ml | | | | |
| 3 | 2021 May 10 | 20:30 | Beer (5%) | 2.0 | Friend | Pub | Pleasant |
| | | | 500 ml | | | | |
| 4 | 2021 May 10 | 21:30 | Shochu (25%) | 1.8 | No one | Home | Calm |
| | | | 90 ml | | | | |
| 5 | 2021 May 10 | 21:45 | Shochu (25%) | 1.8 | No one | Home | Calm |
| | | | 90 ml | | | | |
| 6 | 2021 May 10 | 22:00 | Shochu (25%) | 1.8 | No one | Home | Calm |
| | | | 90 ml | | | | |
| 7 | 2021 May 12 | 20:30 | Beer (5%) | 2.0 | No one | Home | Calm |
| | | | 500 ml | | | | |
| 8 | 2021 May 13 | 19:10 | Shochu (25%) | 1.8 | No one | Home | Angry |
| | | | 90 ml | | | | |
| 9 | 2021 May 13 | 19:20 | Shochu (25%) | 1.8 | No one | Home | Angry |
| | | | 90 ml | | | | |
| 10 | 2021 May 13 | 20:30 | Shochu (25%) | 1.8 | No one | Home | Angry |
| | | | 90 ml | | | | |
| 11 | 2021 May 13 | 21:30 | Shochu (25%) | 1.8 | No one | Home | Angry |
| | | | 90 ml | | | | |
| 12 | 2021 May 13 | 21:45 | Shochu (25%) | 1.8 | No one | Home | Angry |
| | | | 90 ml | | | | |
| 13 | 2021 May 15 | 19:10 | Shochu (25%) | 1.8 | No one | Home | Angry |
| | | | 90 ml | | | | |
| 14 | 2021 May 15 | 19:20 | Shochu (25%) | 1.8 | No one | Home | Angry |
| | | | 90 ml | | | | |
| 15 | 2021 May 15 | 20:30 | Shochu (25%) | 1.8 | No one | Home | Angry |
| | | | 90 ml | | | | |
| 16 | 2021 May 15 | 21:30 | Shochu (25%) | 1.8 | No one | Home | Angry |
| | | | 90 ml | | | | |
| 17 | 2021 May 15 | 21:45 | Shochu (25%) | 1.8 | No one | Home | Angry |
| | | | 90 ml | | | | |
| 18 | 2021 May 15 | 22:00 | Shochu (25%) | 1.8 | No one | Home | Angry |
| | | | 90 ml | | | | |

### (Scene 6): Analysis Conducted for Each Item of Context Information by System

The management server aggregated the number of drinks based on the inputs by classifying the number of drinks according to the context information. As an example, the number of times of drinking and the like classified according to the context information (on the day of drinking) was aggregated as shown in Table 10 below.

TABLE 10

| Item | | With | Where? | | Physical | | | Quantity | Degree of |
|---|---|---|---|---|---|---|---|---|---|
| ID | Date | whom? | (place) | Mood | condition | Feeling | Sleep | of drinks | satisfaction |
| 1 | 2021 May 10 | Friend | Pub | Pleasant | 2 | 2 | 2 | 6 | 1 |
| 2 | 2021 May 10 | No one | Home | Calm | 2 | 2 | 2 | 5.4 | 2 |
| 3 | 2021 May 12 | No one | Home | Calm | 3 | 3 | 4 | 2 | 3 |
| 4 | 2021 May 13 | No one | Home | Angry | 3 | 3 | 4 | 9 | 4 |
| 5 | 2021 May 15 | No one | Home | Angry | 3 | 3 | 4 | 10.8 | 5 |

The number of times of drinking and the like classified according to the context information (the day after drinking) was aggregated as shown in Table 11, where Table 11 corresponds to Table 10.

TABLE 11

| Item ID | Login date | Physical condition | Feeling | Sleep |
|---|---|---|---|---|
| 1 | 2021 May 10 | 1 | 3 | 3 |
| 2 | 2021 May 10 | 1 | 3 | 3 |
| 3 | 2021 May 12 | 3 | 3 | 4 |
| 4 | 2021 May 13 | 3 | 3 | 4 |
| 5 | 2021 May 15 | 3 | 3 | 4 |

Then, the management server further aggregated the quantity of drinks for each category of the context information as shown in Table 12 below.

TABLE 12

| | Context | Quantity |
|---|---|---|
| Context item | category | of drinks |
| With whom? | Friend | 6.0 |
| | No one | 6.8 |
| Where? | Pub | 6.0 |
| | Home | 6.8 |
| Feeling | Pleasant | 6.0 |
| | Calm | 3.7 |
| | Angry | 9.9 |

When the quantity of drinks was aggregated/averaged for each item of the context information as shown in Table 12, the average number of drinks was the largest when “emotion” was “angry” (9.9 drinks). Based on the aggregation results like this, the management server can determine that the amount of drinking of the user increases significantly when the feeling of the user is “angry.” Besides, aggregation may be performed by paying attention to another category. For example, aggregation may be performed based on “the number of places where the user drank in a day” or “the emotion (feeling) the user had when starting drinking.” By performing aggregation in this way, it may be possible to find tendencies as follows: “the amount of drinking tends to increase when the user drinks in two or more places in a day” or “the amount of drinking tends to increase when the emotion at the start of drinking is “angry.””

According to another embodiment of the present invention, aggregation may be performed based on another combination of context information. For example, regarding another combination of context information, a combination of “drinking alone (without any companion), drinking at home, and being angry” could result in a numerical value of 10.8 drinks, which is larger than 9.9 drinks listed above.

According to another embodiment of the present invention, aggregation of context information and the like can be performed on an audience by audience basis rather than on a user by user basis.

The management server can feed back various aggregation results and analysis results to the user in this way. The management server can provide feedback to the user in response to a request from the user as well as with any timing set on the amount-of-drinking management application except during recording of drinking.

### (Scene 7): Checking of Drinking Tendency and the Like Done by User

At the time of application login on the day after drinking, user A made a request to the management server to check to see under what circumstances the amount of drinking tends to increase. Because analysis results were able to be fed back to the user under the condition that data on the user had been accumulated, for example, for seven days including alcohol-free days, the management server made sure that seven days or more of data on the user had been accumulated and then fed back analysis results to the user.

Note that “for seven days” is only an example, and another number of days may be used.

User A opened a feedback screen (not shown). The screen contained drinking tendency scores in different contexts in descending order of drinking tendency, where the contexts were: “Where?,” “With whom,” “With what feeling?,” “When did you start drinking (time) ?,” and “When did you start drinking (circumstance) ?”.

By seeing the scores, user A recognized that the amount of drinking tends to increase when user A drinks with a “friend” in a “pub” or the like by feeling “angry.”

FIG. 11 illustrates an example of a screen that shows high-ranking contexts in terms of drinking tendency to the user in an easy-to-understand manner. In FIG. 11, a screen 1100 displays a title 1110, display fields 1120, 1130, and 1140 of context information on a category by category basis as well as an analysis result display field 1150. The context information category 1120 displays places 1121 and 1122 remaining in the user's action history, and the place 1121 associated with a higher drinking tendency is highlighted. The context information category 1130 displays companion types 1131 and 1132 remaining in the user's action history and the companion type 1131 associated with a higher drinking tendency is highlighted. The context information category 1140 displays the user's feelings 1141 and 1142 remaining in the user's action history and the feeling 1141 associated with a higher drinking tendency is highlighted.

The analysis result display field 1150 displays the context 1151, which is a factor that influences drinking the most.

### (Scene 8): Alert Issued During Recording of Drinking on Another Day

User A drank further on the next day. Before drinking, user A entered “home” as the place of drinking, then the system provided feedback: “You tend to drink more at home than elsewhere. Today you may probably drink more than usual. Be careful.” Viewing the feedback, user A recognized that drinking “at home” increases the amount of drinking, and started drinking at 19 o'clock by pouring a little less amount of beer per glass than usual.

FIG. 12 shows an example of alert display resulting from the user's record input before drinking (or during drinking). In FIG. 12, a screen 1200 displays a drinking date display field 1210, a current quantity-of-drink (and day's target upper limit quantity-of-drink) display field 1220, a system alert display field 1230, display fields 1241 and 1242 of a drink input record from the start of drinking, and a drink input record start button 1250. As also described in the above scenario, by viewing display content of the alert display field 1230, user A was able to start drinking by slowing down the pace of drinking.

### (Scene 9): User Checking Relationship and the Like Between Drinking and Physical Condition

On another day, wanting to know the relationship between his/her own drinking and physical condition, user A made an inquiry to the management server. The management server transmitted the user's recent physical condition history and an amount-of-drinking list on days previous to the respective recent days to the user terminal. By checking the information, user A was able to understand the relationship between drinking and physical condition.

FIG. 13 shows an example of information display that suggests a relationship between the user's drinking and physical condition. In FIG. 13, a screen 1300 displays a message 1310, a recent physical condition history display field 1320, and an amount-of-drinking display field 1330 for days previous to the respective recent days. As shown in FIG. 13, by checking this screen, user A can intuitively and comprehensively grasp the relationship between drinking and physical condition.

### (Variation of Present Invention: Stand-Alone Operation on User Terminal)

According to the embodiment described in detail up to now, the amount-of-drinking management system according to an embodiment of the present invention includes the management server 11 in its configuration. However, the present invention is not restricted to this, and all processes may be designed to be performed on the user terminal as long as there is no logical contradiction. In that case, the device that implements an embodiment of the present invention will be an amount-of-drinking management apparatus (terminal device).

Thus, an example of alternative processes on an amount-of-drinking management apparatus according to another embodiment of the present invention will be described below with reference to FIGS. 5 and 7 to 9.

First, in FIG. 5, the management server is omitted. Then, the processes of steps S504, S509, S512, S515, and S518 are performed on the user terminal of each user, as an example. In order for the doctor's terminal to refer to user data and the like (step S506), either the doctor's terminal accesses the user terminal and refers to the user data individually, or the user data and the like are transmitted to the doctor's terminal as required, allowing the doctor's terminal to refer to the user data.

The processes (steps S701 to S709, steps S801 to S807, and steps S901 to S907) in FIGS. 7 to 9 are also performed on the user terminal. In this case, the user terminal operates stand-alone, and thus can avoid handling data (information about the number of subjects or other user information) other than data of the user terminal.

Whereas embodiments of the amount-of-drinking management system and the like have been described based on specific examples, in addition to a method or program for implementing a system or an apparatus, embodiments of the present invention can be provided in the form of a storage medium (as an example, an optical disk, a magneto-optical disk, a CD-ROM, a CD-R, a CD-RW, a magnetic tape, a hard disk, or a memory card) or the like storing the program.

Implementation form of the program is not limited to object code compiled by a compiler or an application program such as program code executed by an interpreter, and may be a program module or the like incorporated in an operating system.

Furthermore, it is not always necessary that all processes of the program will be performed only by a CPU on a control board, and part or all of the processes may be performed as required by an expansion board added to the control board or by another processing unit (e.g., DSP) mounted on an expansion unit.

All the components described in the present specification (including Claims, Abstract, and Figures) and/or all the steps of all the disclosed methods or processes can be used in any combination except when features of the components are mutually exclusive.

Also, each of the features described in the present specification (including Claims, Abstract, and Figures) can be substituted with an alternative feature serving the same purpose, an equivalent purpose, or a similar purpose unless denied explicitly. Therefore, each of the disclosed features is only a comprehensive series of identical or equivalent features unless denied explicitly.

Furthermore, the present invention is not restricted to any of the concreate configurations of the embodiments described above. The present invention can be extended to all the novel features or combinations thereof or steps of all the novel methods or processes, or combinations thereof, where the novel features, methods, and processes have been described in the present specification (including Claims, Abstract, and Figures).

### REFERENCE SIGNS LIST

10 Amount-of-drinking management system
11 Management server
12 Smart phone or tablet PC (one form of user terminal or the like)
13 Cell phone (one form of user terminal or the like)
14 to 16 PC (one form of user terminal or the like)
17 to 18 Smart phone or tablet terminal (one form of user terminal or the like)
37, 38 Communications line
39 Public line (private line, the Internet, or the like)

1. A method performed on a system that includes a user terminal and a management server configured to be connectable from the user terminal, the method comprising:
accepting information about drinking of a user on the user terminal;
transmitting the information accepted on the user terminal to the management server;
aggregating and/or analyzing information received from the user terminal, on the management server; and
feeding back results of the aggregation and/or the analysis to the user terminal, wherein
the information about the drinking of the user includes at least information about an amount of drinking of the user and context information.

2. The method according to claim 1, wherein the context information includes at least one of items: with whom, where, with what feeling, when does the user starts drinking, and under what circumstances.

3. The method according to claim 1, wherein the aggregation and/or the analysis include(s) aggregating and/or analyzing an amount of drinking for each item of context information contained in information received by the management server.

4. The method according to claim 1, wherein the accepting information about drinking of a user on the user terminal is carried out with one or more of timings of just before drinking, during drinking, just after drinking, or after drinking of the user.

5. The method according to claim 1, wherein feeding back results to the user terminal is carried out with a timing with which the user makes a request to the management server or with a predetermined timing.

6. The method according to claim 3, wherein the aggregation and/or the analysis include(s) aggregating and/or analyzing amounts of drinking for each of two or more items of context information in information received by the management server, based on a combination of the two or more items of context information.

7. The method according to claim 1, wherein in addition to aggregation and/or analysis based on information about the user received by the management server, aggregation and/or analysis are/is conducted based on information about a plurality of users other than the user.

8. The method according to claim 1, wherein feeding back results to the user terminal is carried out under a condition that the user registers predetermined days of data in the management server.

9. A system comprising:
a user terminal; and
a management server configured to be connectable from the user terminal,
wherein the user terminal accepts information about drinking of a user and transmits the accepted information to the management server,
the management server aggregates and/or analyzes information received from the user terminal, and feeds back results of the aggregation and/or the analysis to the user terminal, wherein
information about drinking of the user includes at least information about an amount of drinking of the user and context information.

10. The system according to claim 9, wherein the context information includes at least one of items: with whom, where, with what feeling, when does the user starts drinking, and under what circumstances.

11. The system according to claim 9, wherein the aggregation and/or the analysis include(s) aggregating and/or analyzing an amount of drinking for each item of context information contained in information received by the management server.

12. A terminal device, which is a user terminal device that accepts information about drinking of a user and records the accepted information, wherein:
the user terminal device aggregates and/or analyzes the recorded information, and feeds back results of the aggregation and/or the analysis via a display on the user terminal, wherein
information about drinking of the user includes at least information about an amount of drinking of the user and context information.

13. The terminal device according to claim 12, wherein the context information includes at least one of items: with whom, where, with what feeling, when does the user starts drinking, and under what circumstances.

14. The terminal device according to claim 12, wherein the aggregation and/or the analysis include(s) aggregating and/or analyzing an amount of drinking for each item of context information in the recorded information.

15. A program run on a system that includes a user terminal and a management server configured to be connectable from the user terminal,
the program causing the user terminal to:
accept information about drinking of a user; and
transmit the accepted information to the management server,
the program causing the management server to:
aggregate and/or analyze information received from the user terminal; and
feed back results of the aggregation and/or the analysis to the user terminal, wherein
information about drinking of the user includes at least information about an amount of drinking of the user and context information.

16. The program according to claim 15, wherein the context information includes at least one of items: with whom, where, with what feeling, when does the user starts drinking, and under what circumstances.

17. The program according to claim 15, wherein the aggregation and/or the analysis include(s) aggregating and/or analyzing an amount of drinking for each item of context information contained in information received by the management server.
